(19) United States
(12) Patent Application Publication
(10) Pub. No.: US 2019/0201587 A1
(43) Pub. Date: Jul. 4, 2019

(54) IRON-BASED BIODEGRADABLE COMPONENT APPLICATIONS THEREOF AND METHOD FOR FABRICATING THE SAME

(71) Applicant: INDUSTRIAL TECHNOLOGY RESEARCH INSTITUTE, Hsinchu (TW)

(72) Inventors: Tai-Sheng CHEN, Kaohsiung City (TW); Hong-Jen LAI, Hsinchu City (TW); Ming-Sheng LEU, Zhudong Township (TW); Yion-Ni LIU, Hsinchu City (TW); Jia-Jen CHANG, Huwei Township (TW); Hong-Bin WANG, Taipei City (TW)

(73) Assignee: INDUSTRIAL TECHNOLOGY RESEARCH INSTITUTE, Hsinchu (TW)

(21) Appl. No.: 15/856,844

(22) Filed: Dec. 28, 2017

(51) Int. Cl. A61L 27/58; A61L 27/04; A61L 27/10; A61C 8/00; A61B 17/68; A61F 2/00; A61F 2/24

(52) U.S. Cl. CPC A61L 27/58; A61L 27/042; A61L 27/10; A61L 27/047; A61B 2017/00004; A61B 17/68; A61F 2/0059; A61F 2/24; A61C 8/0012

(57) ABSTRACT

An iron-based biodegradable component includes an iron element of 60 to 99.5 parts by weight and modifying material of 0.5 to 40 parts by weight, wherein the modifying material includes at least one of a zinc element of 0.1 to 5 parts by weight and a biodegradable ceramic material of 0.1 to 40 parts by weight.

### TECHNICAL FIELD

The disclosure relates in general to a biodegradable component and the applications thereof as well as the method for fabricating the same and more particularly to an iron-based biodegradable component and the applications thereof as well as the method for fabricating the same.

### BACKGROUND

As the increase of the share of older individuals in a society due to fertility declines and rising life expectancy, population aging is an irreversible global trend, the medical expenses will grow continuously and the demand for medical implants will also increase. Typical medical implants, such as bone nail and bone plate used in orthopedic surgery, are formed of metals (such as stainless steel, cobalt chromium alloy, titanium and titanium alloy) and have the advantages of high strength, high toughness, high fatigue resistance, high corrosion resistance, high plasticity, high workability and high economy. However, the metal medical implant does not degrade after being implanted in a human body, but has the potential risk of infection. Normally, after the wound heals, a second surgery is required to remove the metal medical implant from the body.

The second surgery for removing the medical implant has the clinical risk of causing complications and damaging nerves. Therefore, a new technology for fabricating a medical implant using biodegradable polymers such as polylactic acid (PLA), polyglycolic acid (PGA), polycyanoacrylate (PACA) is provided in response to the need. The medical implant formed of polymers can be absorbed by the human body and there is no need to perform a second surgery to remove it from the body, hence avoiding causing extra risks and damages to the patient. However, the medical implant formed of biodegradable polymer materials still has the problems that lacking sufficient mechanical strength, having poor mechanical properties and quick degradation rate and being unable to bear an excessive stress.

To resolve the problem of lacking sufficient mechanical strength, a technology of fabricating a medical implant made of a biodegradable component using metal-based material, such as magnesium, iron, manganese or an alloy thereof, as the base is provided. However, the medical implant formed of a magnesium-based biodegradable component has a quicker degradation rate in the human body result in the mechanical strength is hard to maintain. The medical implant formed of an iron-based biodegradable component has a slower degradation rate and the stay time in the body is hard to adjust and control. Moreover, when the proportion of the manganese element of iron-based biodegradable component is too high, biotoxicity may easily be generated.

Therefore, it has become a prominent task for the industries to provide an advanced iron-based biodegradable component and a method for fabricating the same.

### SUMMARY

According to one embodiment, an iron-based biodegradable component is disclosed. The iron-based biodegradable component includes an iron element (Fe) of 60˜99.5 parts by weight and a modifying material of 0.5˜40 parts by weight. The modifying material includes at least one of a zinc element (Zn) of 0.1˜5 parts by weight and a biodegradable ceramic material of 0.1˜40 parts by weight.

According to another embodiment, a biodegradable medical implant containing the iron-based biodegradable component as described above is disclosed.

A method for fabricating a biodegradable medical implant is provided. The fabricating method includes the following steps: Firstly, the iron-based biodegradable component as described above is provided, wherein the iron-based biodegradable component includes a plurality of iron metal particles and a plurality of modifying particles. Then, the iron metal particles and the modifying particles of the iron-based biodegradable component are fully mixed.

According to the above embodiment, an iron-based biodegradable component and the applications as well as a method for fabricating the same are provided in the present specification. The iron-based biodegradable component uses iron as a base material and further includes other modifying materials, such as a zinc-containing material, a biodegradable ceramic material or a combination of the zinc-containing material and the biodegradable ceramic material. The iron element has 60˜99.5 parts by weight; the zinc element has 0.1˜5 parts by weight; the biodegradable ceramic material has 0.1˜40 parts by weight. The above materials can be mixed, melted, hot-pressed or sintered to form a biodegradable medical implant with various structures such as powder, plate, block or 3D assembly. The biodegradable medical implant can be used as a dental implant, an orthopedic implant, a cardiac implantation or a plastic surgical implant to resolve the problems of the generally known medical implants that cannot be absorbed by human body, lack sufficient mechanical strength and have a biodegradation rate hard to adjust or control.

### BRIEF DESCRIPTION OF THE DRAWINGS

The above and other aspects of the invention will become better understood with regard to the following detailed description of the preferred but non-limiting embodiment (s). The following description is made with reference to the accompanying drawings.

FIG. 1A is a flowchart of a method for fabricating a biodegradable medical implant according to an embodiment of the present specification;

FIG. 1B is a partial structure of a process for fabricating a biodegradable medical implant using the method of FIG. 1A;

FIG. 2A is a flowchart of a method for fabricating a biodegradable medical implant according to another embodiment of the present specification;

FIGS. 2B˜2C are partial structures of a process for fabricating a biodegradable medical implant using the method of FIG. 2A;

FIG. 3A is a flowchart of a method for fabricating a biodegradable medical implant according to an alternate embodiment of the present specification;

FIGS. 3B˜3C are partial structures of a process for fabricating a biodegradable medical implant using the method of FIG. 3A;

FIG. 4 is a perspective diagram of the structure of a biodegradable medical implant according to an alternate embodiment of the present specification;

FIG. 5A is a flowchart of a method for fabricating a biodegradable medical implant according to another alternate embodiment of the present specification; and

FIGS. 5B˜5C are partial structures of a process for fabricating a biodegradable medical implant using the method of FIG. 5A.

### DETAILED DESCRIPTION

The present specification provides an iron-based biodegradable component and a method for fabricating the same capable of resolving the problems that the generally known medical implants cannot be absorbed by human body, lack sufficient mechanical strength and have a biodegradation rate hard to adjust or control. For the object, technical features and advantages of the embodiment of the present specification to be more easily understood, a number of exemplary embodiments are disclosed below with detailed descriptions and accompanying drawings.

It should be noted that these embodiments are for exemplary and explanatory purposes only, not for limiting the scope of protection of the invention. The invention can be implemented by using other features, elements, methods and parameters. The preferred embodiments are merely for illustrating the technical features of the invention, not for limiting the scope of protection. Anyone skilled in the technology field of the invention will be able to make suitable modifications or changes based on the specification disclosed below without breaching the spirit of the invention. Designations common to the accompanying drawings are used to indicate identical or similar elements.

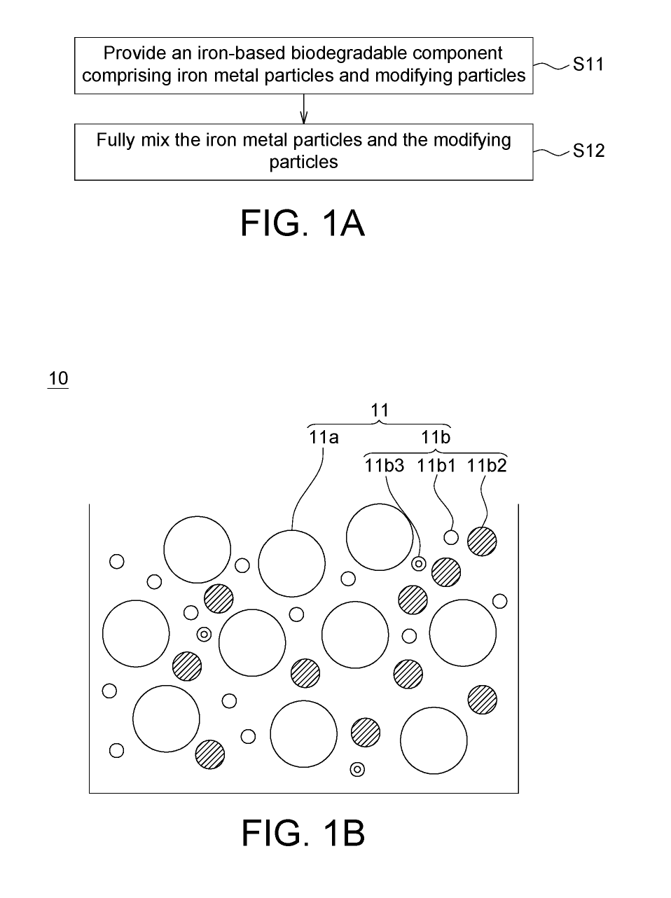

Refer to FIGS. 1A˜1B. FIG. 1A is a flowchart of a method for fabricating a biodegradable medical implant 10 according to an embodiment of the present specification. FIG. 1B is a partial structure of a process for fabricating a biodegradable medical implant 10 using the method of FIG. 1A. In the present embodiment, the method for fabricating a biodegradable component 10 includes the following steps:

Firstly, an iron-based biodegradable component 11 is provided (step S11). In some embodiments of the present specification, the iron-based biodegradable component 11 may include a plurality of iron metal particles 11a and a plurality of modifying particles 11b. The modifying particles 11b can be formed of zinc-containing material particles 11b1 only, the biodegradable ceramic material particles 11b2 only, or both the zinc-containing material particles 11b1 and the biodegradable ceramic material particles 11b2. In the iron-based biodegradable component 11, the iron element has 60˜99.5 parts by weight; the zinc element has 0.1˜5 parts by weight; and the biodegradable ceramic material has 0.1˜40 parts by weight.

The zinc-containing material particles 11b1 can be formed of zinc metal particles, zinc oxide particles or a combination thereof. The biodegradable ceramic material particles 11b2 can be formed of materials such as hydroxyapatite (HA), tricalcium phosphate (TCP), titanium oxide, aluminum oxide, silicon oxide, zirconia oxide, or a combination thereof. Besides, the modifying particles 11b may further include at least one kind of magnesium-containing particles 11b3, such as the magnesium metal particles. In some embodiments of the present specification, the magnesium element (Mg) has 0.1˜5 parts by weight.

In the present embodiment, the biodegradable component 11 may include a plurality of iron metal particles 11a with an average particle size of 1˜500 micrometers (μm) and a plurality of modifying particles 11b with an average particle size of 0.1˜500 μm. The modifying particles 11b include a plurality of zinc metal particles (the zinc-containing material particles 11b1), a plurality of tricalcium phosphate particles (the biodegradable ceramic material 11b2) and a plurality of magnesium metal particles (the magnesium-containing particles 11b3). The iron metal particles have 85 parts by weight; the zinc metal particles have 3 parts by weight; the tricalcium phosphate particles have 10 parts by weight; magnesium metal particles have 1 parts by weight.

However, the materials of the biodegradable component 11 are not limited thereto. In some other embodiments of the present specification, the biodegradable component 11 may only include a plurality of iron metal particles 11a, a plurality of zinc-containing material particles 11b with an average particle size of 0.1˜500 μm and a plurality of tricalcium phosphate particles (the biodegradable ceramic material 11b2). The zinc-containing material particles 11b1 may have 1˜5 parts by weight of the biodegradable component 11; and the tricalcium phosphate (the biodegradable ceramic material 11b2) particles may have 5˜30 parts by weight of the biodegradable component 11.

In some other embodiments of the present specification, the biodegradable component 11 may only include a plurality of iron metal particles 11a and a plurality of zinc-containing material particles 11b1 with an average particle size of 0.1˜500 μm. The zinc-containing material particles 11b1 may have 0.5˜5 parts by weight of the biodegradable component 11. In a practical embodiment, the iron metal particles have 95 parts by weight; and the zinc-containing material particles 11b1 have 5 parts by weight 5. In another practical embodiment, iron metal particles 11a have 90 parts by weight; and the zinc-containing material particles 11b1 have 3 parts by weight.

In some alternate embodiments of the present specification, the biodegradable component 11 may only include a plurality of iron metal particles 11a and a plurality of tricalcium phosphate particles (the biodegradable ceramic material 11b2). The tricalcium phosphate particles (the biodegradable ceramic material 11b2) may have 1˜40 parts by weight of the biodegradable component 11. In a practical embodiment, the iron metal particles have 90 parts by weight; and the tricalcium phosphate particles (the biodegradable ceramic material 11b2) have 10 parts by weight.

Then, the iron metal particles 11a and the modifying particles 11b are fully mixed (step S12) to complete the preparation of the biodegradable medical implant 10 (see FIG. 1B) with a powder structure. In some embodiments of the present specification, the biodegradable medical implant 10 can be used as a filling of periodontal tissue (alveolar bone), bone or connective tissue (such as skin) in dental implantation, orthopedic surgery, or plastic surgical surgery. In some other embodiments of the present specification, the biodegradable medical implant 10 with a powder structure can also be used as an initial material for fabricating a biodegradable medical implant with different structure. The steps and materials for fabricating a biodegradable medical implant with different structures are disclosed below:

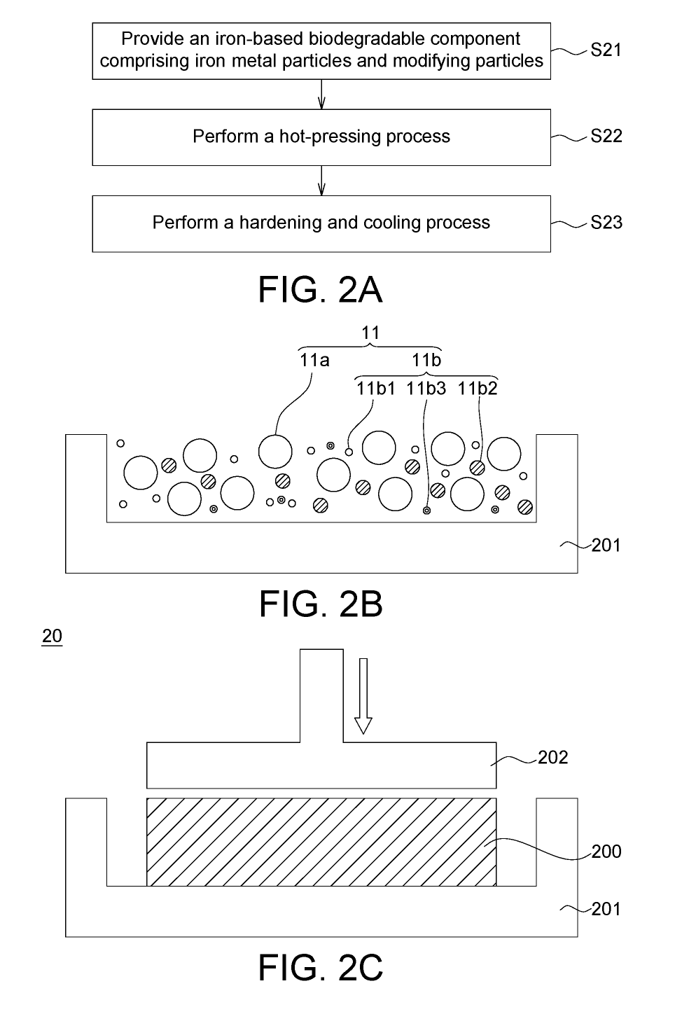

Refer to FIGS. 2A˜2B, FIG. 2A is a flowchart of a method for fabricating a biodegradable medical implant 20 according to another embodiment of the present specification. FIGS. 2B˜2C are partial structures of a process for fabricating a biodegradable medical implant 20 using the method of FIG. 2A. In the present embodiment, the method for fabricating a biodegradable component 20 includes the following steps:

Firstly, the iron-based biodegradable component 11 of FIG. 1B is provided (step S21). The components and formation steps of the biodegradable component 11 are already disclosed in FIGS. 1A˜1C and therefore are not redundantly repeated. Then, a hot-pressing process is performed (step S22). The fully mixed biodegradable component 11 is infused to a hot-pressing mold 201 (see FIG. 2B). The heating temperature and time are controlled for allowing the biodegradable component 11 to be melted. Then, a compression molding is performed, in which the melted biodegradable component 11 is compression molded by a mechanical stress using a tool 202. After the biodegradable component 11 is hardened and cooled (step S23), a plate 200 with a flake structure (see FIG. 2C) is formed. In some embodiments of the present specification, the hot-pressing process has an operating temperature of 500˜800° C.

Subsequently, a downstream process, such as mechanical processing, cutting, stamping, drilling, or welding (not illustrated) are performed, and the biodegradable medical implant 20 used in orthopedic surgery, plastic surgical surgery or dental implantation can be thus formed. In some embodiments of the present specification, the biodegradable medical implant 20 can be an artificial bone plate used in orthopedic surgery.

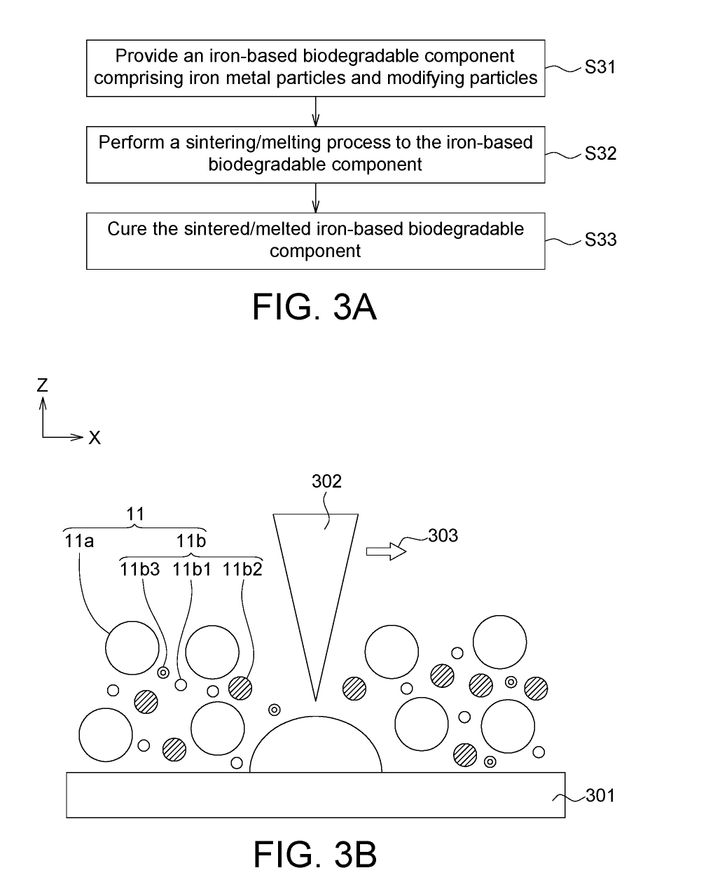

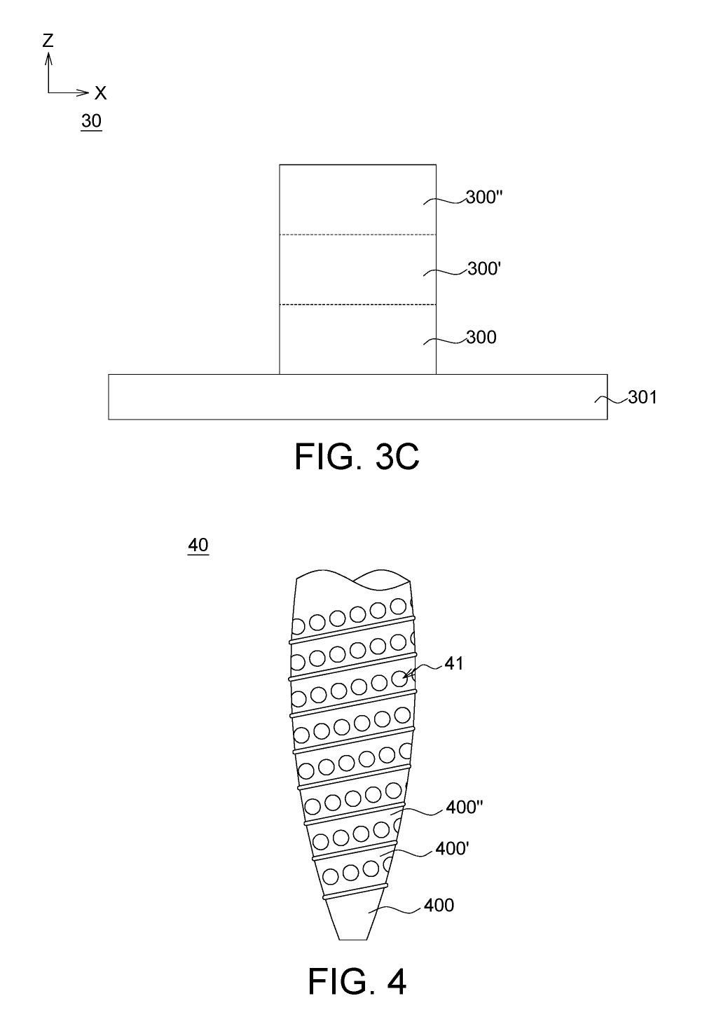

Refer to FIGS. 3A˜3C. FIG. 3A is a flowchart of a method for fabricating a biodegradable medical implant 30 according to an alternate embodiment of the present specification. FIGS. 3B˜3C are partial structures of a process for fabricating a biodegradable medical implant 30 using the method of FIG. 3A. In the present embodiment, the method for fabricating a biodegradable component 30 includes the following steps:

Firstly, an iron-based biodegradable component 11 is provided on a substrate 301 (step S31). In the present embodiment, the iron-based biodegradable component 11 can be provided by placing the fully mixed iron-based biodegradable component 11 on a surface of the substrate 301. The components and formation steps of the iron-based biodegradable component 11 are already disclosed in FIGS. 1A˜1C and therefore are not redundantly repeated.

Then, a sintering/melting process is performed to the iron-based biodegradable component 11 (step S32). In some embodiments of the present specification, the sintering/melting process may include providing a focused energy beam 302 for sintering/melting the iron-based biodegradable component 11 along a default scan path 303 (see FIG. 3B). In the present embodiment, the sintering/melting process can adopt a laser beam (focused energy beam 302) of 200˜340 watts (W) for sintering/melting the powder particles of the iron-based biodegradable component 11 along the X axis at a scanning speed of 1500˜4500 millimeters per second (mm/s).

Then, the sintered/melted iron-based biodegradable component 11 is cured to form at least one lamination layer 300 on a surface 11a of the substrate 11 (step S33). In some embodiments of the present specification, the step of curing the sintered/melted iron-based biodegradable component 11 may include performing an annealing treatment to the sintered/melted iron-based biodegradable component 11 in an air atmosphere.

Then, the above steps S1˜S3 are repeated to form a plurality of lamination layers, such as lamination layers 300′ and 300″, stacked on the lamination layer 300 to form a columnar block with a 3D structure (see FIG. 3C). Subsequently, a downstream process, such as mechanical processing, cutting, stamping, drilling, or welding (not illustrated) are performed, and the biodegradable medical implant 30 used in orthopedic surgery, plastic surgical surgery or dental implantation can be thus formed. In some embodiments of the present specification, the biodegradable medical implant 30 can be an artificial bone nail used in orthopedic surgery.

Referring to FIG. 4, a perspective diagram of the structure of a biodegradable medical implant 40 according to an alternate embodiment of the present specification is shown. The materials and fabricating method of the biodegradable medical implant 40 are similar to that of FIGS. 3A˜3C except that: a predetermined shape of the biodegradable medical implant 40 can be obtained by adjusting the scan path in each sintering/melting process for forming each lamination layer of the biodegradable medical implant 40. As a result, the cross-sectional profile of each lamination layer can be altered according to the scan path, and the 3D structure of the biodegradable medical implant 40 that is formed by stacking the lamination layers can thus have the predetermined shape. In the present embodiment, by altering the shapes and profiles of the lamination layers 400, 400′ and 400″, the lamination layers 400, 400′ and 400″ can form a 3D assembly with 3D threads and a tapered tubular structure. The biodegradable medical implant 40 can be used as an artificial bone nail in orthopedic surgery. Moreover, at least one hole 41 can be formed at particular position of the biodegradable medical implant 40 to accelerate the biodegradation rate at the said position.

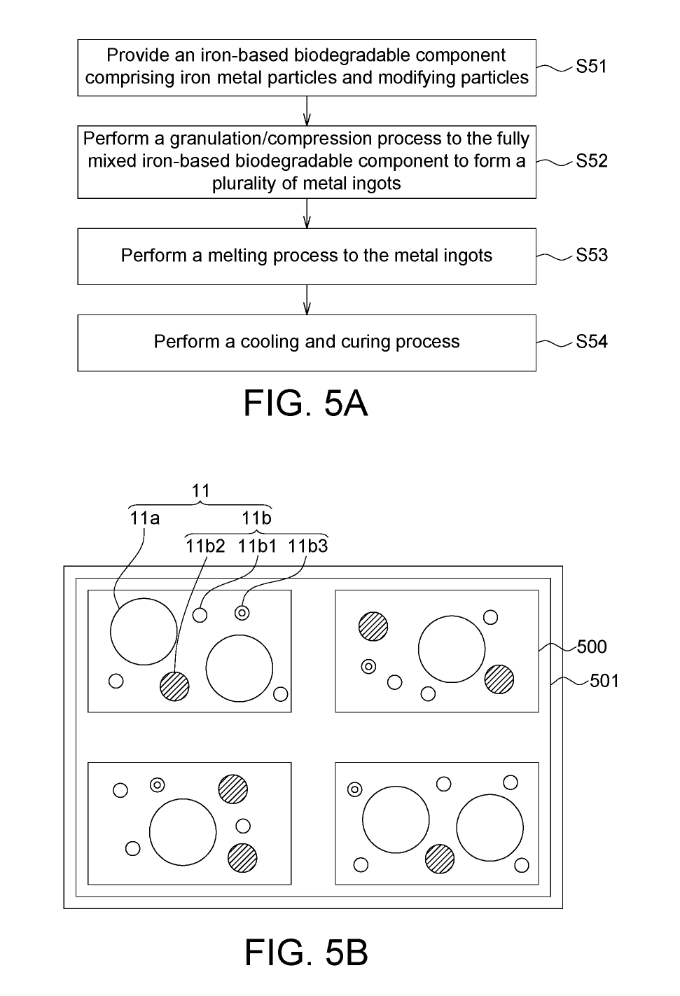

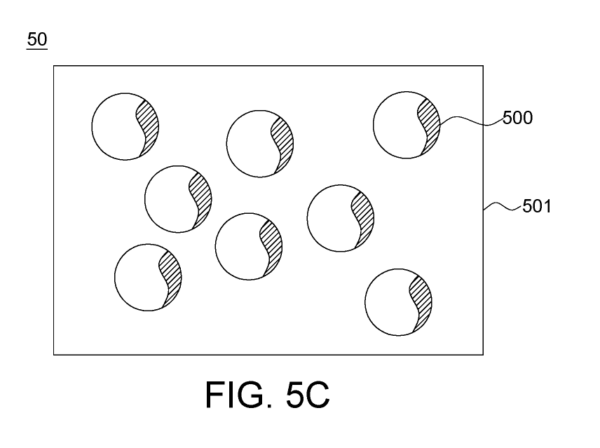

Refer to FIGS. 5A˜5C, FIG. 5A is a flowchart of a method for fabricating a biodegradable medical implant 50 according to another alternate embodiment of the present specification. FIGS. 5B˜5C are partial structures of a process for fabricating a biodegradable medical implant 50 using the method of FIG. 5A. In the present embodiment, the method for fabricating a biodegradable component 50 includes the following steps:

Firstly, an iron-based biodegradable component 11 is provided (step S51). In the present embodiment, the iron-based biodegradable component 11 is provided by performing a granulation/compression process to the fully mixed iron-based biodegradable component 11 to form a plurality of metal ingots 500 (step S52). Then, the metal ingots 500 are placed in a melting furnace 501 (see FIG. 5B). The components and formation steps of the iron-based biodegradable component 11 are already disclosed in FIGS. 1A˜1C and therefore are not redundantly repeated.

Then, a melting process is performed to the blocky metal ingots 500 (step S53). In some embodiments of the present specification, the melting process has an operating temperature of 1000˜1500° C. at which the iron metal particles 11a, the zinc-containing material particles 11b1, the biodegradable ceramic material particles 11b2 and the magnesium-containing particles 11b3 of the iron-based biodegradable component 11 can be melted and solidifying. Then, the melting clumps with a powder structure (see FIG. 5C) can be cooled and cured (step S54) to complete the preparation of the biodegradable medical implant 50.

In some embodiments of the present specification, the biodegradable medical implant 50 can be used as a filling of periodontal tissue (alveolar bone), bone or connective tissue (such as skin) in dental implantation, orthopedic surgery, or plastic surgical surgery. In some other embodiments of the present specification, the biodegradable medical implant 50 with a powder structure can also be used as an initial material in other processing method (such as the hot-pressing process of FIG. 2B or the sintering/melting process of FIG. 3B) for fabricating a biodegradable medical implant with different structure.

A biodegradation test and an acute toxicity test (e.g. assessing cell metabolic activity by a MTT assay) based on ISO 10993-5, “the series of standards for the biological evaluation of medical devices” compiled by the International Organization for Standardization, are performed to estimate the biodegradability and the acute toxicity of the iron metal particles 11a, the zinc-containing material particles 11b1, the biodegradable ceramic material particles 11b2, the magnesium-containing particles 11b3 and the biodegradable medical implants 10, 20, 30 and 50 fabricated in the above embodiments. A tension test according to the ASTM E8 testing method formulated by the American Society of Testing and Materials (ASTM) is performed to measure the tensile strength of the biodegradable medical implants 10, 20, 30 and 50.

In according to the test results, the iron metal particles 11a, the zinc-containing material particles 11b1, the biodegradable ceramic material particles 11b2, the magnesium-containing particles 11b3 and the biodegradable medical implants 10, 20, 30 and 50 do not adversely affect the cell lines, the cell survival rate or cell inhibition percentage may be greater than about 70%. The biodegradable medical implants 10, 20, 30 and 50 having a biodegradation rate larger than 0.2 mm/year can gradually degrade and eventually vanish in the human body. The biodegradable medical implants 10, 20, 30 and 50 have a yield strength larger than 200 MPa and therefore have a mechanical strength suitable for human skeleton.

In some embodiments of the present specification, the oxidation of the metal elements of the iron-based biodegradable component 11 can be controlled by adjusting the proportions of the iron metal particles 11a, the zinc-containing material particles 11b1, the biodegradable ceramic material particles 11b2 and the magnesium-containing particles 11b3 as well as by controlling the air atmosphere in the hot-pressing process, the sintering/melting process or the melting process of the method for fabricating the biodegradable medical implants 20, 30, 40 and 50. Thus, the proportions of the iron element, the zinc element and/or the oxygen element of the biodegradable medical implant 20, 30, 40 and 50 can be adjusted and the object of adjusting the biodegradation rate of the biodegradable medical implants 20, 30, 40 and 50 can therefore be achieved.

According to the above embodiments, the present specification provides an iron-based biodegradable component and the applications as well as a method for fabricating the same. The iron-based biodegradable component mainly uses iron as the base material and further includes other modifying materials, such as a zinc-containing material, a biodegradable ceramic material or a combination of the zinc-containing material and the biodegradable ceramic material. The iron element has 60˜99.5 parts by weight; the zinc element has 0.1˜5 parts by weight; and the biodegradable ceramic material has 0.1˜40 parts by weight. The above materials can be mixed, melted, hot-pressed or sintered to form a biodegradable medical implant with various structures such as powder, plate, block or 3D assembly. The biodegradable medical implant can be used as a dental implant, an orthopedic implant, a cardiac implant or a plastic surgical implant to resolve the problems of the generally known medical implants that cannot be absorbed by human body, lack sufficient mechanical strength and have a biodegradation rate hard to adjust or control.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. An iron-based biodegradable component, comprising:
an iron element (Fe) of 60 to 99.5 parts by weight; and
a modifying material of 0.5 to 40 parts by weight;
wherein, the modifying material comprises: at least one of a zinc element (Zn) of 0.1 to 5 parts by weight and a biodegradable ceramic material of 0.1 to 40 parts by weight.

2. The iron-based biodegradable component according to claim 1, wherein the biodegradable ceramic material is selected from a group composed of hydroxyapatite (HA), tricalcium phosphate (TCP), titanium oxide, aluminum oxide, silicon oxide, zirconia oxide and any combination thereof.

3. The iron-based biodegradable component according to claim 1, wherein the biodegradable ceramic material is TCP of 0.1 to 10 parts by weight.

4. The iron-based biodegradable component according to claim 2, further comprising magnesium element (Mg) of 0.1 to 5 parts by weight.

5. A biodegradable medical implant, comprising:
an iron-based biodegradable component, comprising:
an iron element (Fe) of 60 to 99.5 parts by weight; and
a modifying material of 0.5 to 40 parts by weight;
wherein, the modifying material comprises: at least one of a zinc element (Zn) of 0.1 to 5 parts by weight and a biodegradable ceramic material of 0.1 to 40 parts by weight.

6. The biodegradable medical implant according to claim 5, wherein the biodegradable ceramic material is selected from a group composed of HA, TCP, titanium oxide, aluminum oxide, silicon oxide, zirconia oxide and any combination thereof.

7. The biodegradable medical implant according to claim 5, wherein the biodegradable ceramic material is TCP of 0.1 to 10 parts by weight.

8. The biodegradable medical implant according to claim 5, wherein the iron-based biodegradable component further comprises magnesium element (Mg) of 0.1 to 5 parts by weight.

9. The biodegradable medical implant according to claim 5, further comprising a plurality of iron metal particles and a plurality of modifying particles formed of the modifying material.

10. The biodegradable medical implant according to claim 5, comprising a plate, a block or a 3D assembly formed of the iron-based biodegradable component.

11. The biodegradable medical implant according to claim 5, being a dental implant, an orthopedic implant, a cardiac implant or a plastic surgical implant.

12. A method for fabricating a biodegradable medical implant, comprising:
providing an iron-based biodegradable component, comprising a plurality of iron metal particles and a plurality of modifying particles formed of a modifying material; and
fully mixing the iron metal particles and the modifying particles.

13. The method according to claim 12, wherein the step of fully mixing the powder comprises:
melting the iron-based biodegradable component; and
curing and molding the melted iron-based biodegradable component.

14. The fabricating method of biodegradable medical implant according to claim 13, wherein the step of melting the iron-based biodegradable component is a melting process, comprising:
performing a granulation/compression process to the iron-based biodegradable component to form a plurality of metal ingots; and
heating the metal ingots at an operating temperature of 800 to 1500° C.

15. The fabricating method of biodegradable medical implant according to claim 13, wherein the step of melting the iron-based biodegradable component is a sintering/melting process.

16. The fabricating method of biodegradable medical implant according to claim 13, wherein the step of curing and molding the melted iron-based biodegradable component comprises a hot-pressing molding process comprising an operating temperature of 600 to 800° C.

17. The fabricating method of biodegradable medical implant according to claim 13, wherein the step of curing and molding the melted iron-based biodegradable component is an annealing treatment.
